(19) United States
(12) Patent Application Publication
(10) Pub. No.: US 2006/0233876 A1
(43) Pub. Date: Oct. 19, 2006

(54) Compositions and methods for reducing cardiovascular morbidity and/or mortality

(76) Inventors: Kenneth Jamerson, West Bloomfield, MI (US); Malcolm MacNab, Far Hills, NJ (US); Bertram Pitt, Ann Arbor, MI (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/969,700

(22) Filed: Oct. 20, 2004

Related U.S. Application Data
(60) Provisional application No. 60/512,634, filed on Oct. 20, 2003.

(51) Int. Cl. A61K 31/704; A61K 31/549; A61K 31/455; A61K 31/401; A61K 9/20

(52) U.S. Cl. 424/464; 514/26; 514/355; 514/423; 514/223.5

(57) ABSTRACT

The present invention is related to a method for reducing cardiovascular morbidity and/or mortality comprising administering a combination comprising an ACE inhibitor and a CCB, specifically benazapril and amlodipine besylate.

### BACKGROUND OF THE INVENTION

Essential hypertension, which affects 600 million people worldwide, is the most prevalent vascular disease in the world. See Martin, Clin Exp Hypertens, Vol. 21, Nos. 5-6, pp. 659-669 (1999). Hypertension is a major risk factor for coronary heart disease (CHD), stroke, heart failure and chronic kidney disease. It is estimated that 35% of atherosclerotic cardiovascular events may be attributable to hypertension. See Kannel, JAMA, Vol. 275, No. 20, pp. 1571-1576 (1996). Because the prevalence, incidence and complications of hypertension increase with advancing age, the impact of hypertension is likely to increase as the population ages.

Blood pressure is directly and continuously related to the risk of stroke and cardiovascular disease. See Collins and MacMahon, Br Med Bull, Vol. 50, No. 2, pp. 272-298 (1994). The higher the blood pressure, the more likely that a cardiovascular event will occur. Epidemiological studies have confirmed that systolic blood pressure (SBP) is a more important risk factor than diastolic blood pressure (DBP). See Stamler, Stamler and Neaton, Arch Intern Med, Vol. 153, No. 5, pp. 598-615 (1993).

Many patients with hypertension have inadequate control of blood pressure. See Berlowitz et al., N Engl J Med, Vol. 339, No. 27, pp. 1957-1963 (1998); and Marques-Vidal and Tuomilehto, J Hum Hypertens, Vol. 11, No. 4, pp. 213-220 (1997). The National Health and Nutrition Examination Survey (NHANES 3) reported that only half of all hypertensive Americans that were receiving treatment had their blood pressure controlled to <140/90 mmHg. See Burt et al., Hypertension, Vol. 25, No. 3, pp. 305-313 (1995). Many reasons exist for inadequate blood pressure control, including poor patient compliance, reluctance of physicians to titrate medication, concerns with adverse events and lack of success with monotherapy. See Berlowitz et al. (1998), supra; and Materson et al., N Engl J Med, Vol. 328, No. 13, pp. 914-921 (1993). Recent studies have shown that most patients require a combination of antihypertensive medications to reach goal blood pressure. See Hansson et al., for the HOT Study Group, Lancet, Vol. 351, No. 9118, pp. 1755-1762 (1998); UK Prospective Diabetes Study Group: UKPDS 38, BMJ, Vol. 317, No. 7160, pp. 703-713 (1998); and ALLHAT Officers and Coordinators for the ALLHAT Collaborative Research Group. The Antihypertensive and Lipid-Lowering Treatment to Prevent Heart Attack Trial, JAMA, Vol. 288, No. 23, pp. 2981-2997 (2002).

Lotrel® (amlodipine and benazepril hydrochloride) is a combination of the ACE-inhibitor benazepril and the dihydropyridine calcium channel antagonist amlodipine. The components of Lotrel® have complementary mechanisms of action with effects on blood pressure reduction, and the combination causes fewer side effects, in particular less edema, than amlodipine alone. See Lotrel® Package Insert, Physician's Desk Reference, 57^{th} edition (2003).

Benazepril, benazeprilat, and their pharmaceutically acceptable salts are disclosed in U.S. Pat. No. 4,410,520 (patent '520), along with pharmaceutically acceptable dosage forms thereof, dosage ranges and suitable routes of administration therewith, and uses therefor, all of which are incorporated herein by reference. Amlodipine and its pharmaceutically acceptable salts are set forth in U.S. Pat. No. 4,572,909 (patent '909), incorporated herein by reference. Pharmaceutically acceptable dosage forms, dosage ranges, suitable routes of administration, and uses of amlodipine and its salts are set out there. U.S. Pat. No. 4,879,303 (patent '303) is directed to the besylate salt of amlodipine, and it too is incorporated herein by reference. More specific dosages, routes of administration, formulations and uses for amlodipine besylate can be found there. An excellent review of amlodipine is Burges et al., Cardiovas Drug Dev, Vol. 8, No. 1, pp. 25-44 (1990). Also, diuretics are the most frequently used drug class in combination therapy.

Hypertensive patients, particularly high-risk hypertensive patients, are extremely vulnerable to cardiovascular morbidity and/or mortality. Accordingly, there is a need for effective compositions and methods which reduce cardiovascular morbidity and/or mortality in hypertensive patients.

### OBJECTS OF THE INVENTION

It is an object of the invention to provide compositions and methods for reducing cardiovascular morbidity and mortality in patients with hypertension, such compositions comprising an angiotensin converting enzyme inhibitor (ACEI) and a calcium channel blocker (CCB). In a preferred embodiment, the patients with hypertension are high-risk hypertensive patients. Preferably, the ACEI is benazepril, benazeprilat, and pharmaceutically acceptable salts thereof and the CCB is amlodipine and pharmaceutically acceptable salts thereof, especially the besylate salt.

Another object of the invention is to provide compositions and methods for reducing cardiovascular morbidity and mortality in patients with hypertension, such compositions comprising an ACEI, a CCB and a diuretic. In a preferred embodiment, the patients with hypertension are high-risk hypertensive patients.

### SUMMARY OF THE INVENTION

Surprisingly, these and other objects of the present invention are accomplished by the compositions and methods of the present invention. In one aspect the present invention is related to a method for reducing morbidity and/or mortality in mammals with hypertension comprising administering to said mammal cotherapy of:
(a) an ACEI selected from benazepril, benazeprilat, and pharmaceutically acceptable salts thereof; and
(b) a CCB selected from amlodipine and pharmaceutically acceptable salts thereof.

In a preferred embodiment, the patients with hypertension are high-risk hypertensive patients and amlodipine is the besylate salt of amlodipine.

Other components may optionally be included as part of the compositions or methods of this invention. When included, such optional components will generally include a diuretic.

### DETAILED DESCRIPTION OF THE INVENTION

More specifically, in one aspect the present invention is related to a method for reducing cardiovascular morbidity and mortality in mammals with hypertension, especially humans, comprising administering
(a) an ACEI selected from the group consisting of benazepril, benazeprilat, and pharmaceutically acceptable salts thereof; and
(b) a CCB selected from the group consisting of amlodipine and pharmaceutically acceptable salts thereof.

In a preferred embodiment, the patients with hypertension are high-risk hypertensive patients and amlodipine besylate is the CCB.

Other components may optionally be included as part of the compositions or methods of this invention. When included, such optional components will generally include a diuretic.

Suitable salts of benazepril and benazeprilat can be found in patent '520, mentioned above. For purposes of the present invention, the hydrochloride salt of the ACEI is most advantageous, with the most preferred specific ACEI compound being be nazepril hydrochloride.

The present invention CCB is limited to amlodipine or its salts, which are set forth in the above cited patent '909, with the most suitable salt being the besylate salt (the subject matter of patent '303).

A diuretic may optionally be included as part of the therapeutic regimen and may similarly be widely selected from among those conventionally known in the art. Useful diuretics include methyclothiazide, hydrochlorothiazide, torsemide, metolazone, furosemide, chlorthalidone, N-(5-sulfamoyl-1,3,4-thiadiazol-2-yl)acetamide, triamterene, chlorothiazide, indapamide, bumetanide, amiloride, spironolactone, bendroflumethiazide, benzthiazide, cyclothiazide, quinethazone, hydroflumethiazide, polythiazide, trichlormethiazide and ethacrynic acid.

While the ACEI and the CCB, and optionally a diuretic, can be administered at different times, they are most preferably administered at the same time. Most conveniently, this is via a single, fixed combination dosage form. However, the ACEI can be administered at times different from the administration of the CCB and the invention benefits still be realized. When administered at different times, the ACEI and the CCB should be given within about 16 hours of each other, preferably within about 12 hours of each other, more preferably within about 8 hours of each other, most preferably within about 4 hours of each other. Of course, these time periods can be extended if the dosage form is one which will “administer” the agents for extended periods.

When the ACEI and the CCB, and optionally a diuretic, are given substantially simultaneously, they may be given by a single fixed combination dosage form or by different dosage forms, whichever is convenient. When given by different dosage forms, it is irrelevant whether the route of administration is the same for each agent or different for each agent. Any route of administration known for the individual agents is acceptable for the practice of the present invention. Most preferably, the agents are given in a fixed combination, or at least substantially simultaneously, i.e., within about one hour of each other. Also, the most suitable dosage form is an oral dosage form, where oral administration is a clinically suitable route.

Dosages of the two agents include all dosages at which the agents are used individually. Typically, the dosage of the ACEI is from about 2 mg to about 80 mg, preferably about 3 mg to about 40 mg, more preferably about 5 mg to about 20 mg (based on benazepril hydrochloride). Generally the dosage of the CCB is about 1 mg to about 20 mg, more preferably about 2 mg to about 10 mg, more preferably about 2.5 mg to about 5 mg (based on amlodipine free base). Corresponding dosages for other salts of amlodipine, for free benazepril and other salts of benazepril, and benazeprilat and its salts will be readily apparent to those of ordinary skill in the art. In each of the dosages set forth here, the range is the acceptable range based an adult mammal of approximately 50 kg to about 70 kg. Modified dosage ranges for mammals of other sizes and stages of development will be apparent to those of ordinary skill. In the practice of the present invention, the weight ratio of the ACEI to CCB (based upon benazepril hydrochloride:amlodipine free base) is from about 0.5:1 to about 10:1, more preferably 1:1 to 8:1. The precise weight ratios when using salts other than those set forth above may change, but only because the corresponding amount of the active agents have different weights. Those of ordinary skill in the art will be able to make the appropriate calculations. Particularly advantageous ratios of benazepril hydrochloride:amlodipine free base are 1:1, 2:1, 4:1 and 8:1.

Benazepril and amlodipine are physically incompatible substances. Hence, if incorporated into a single dosage form they must be kept physically separated. This may be accomplished in any of the myriad ways known in the art, such as bi-layered tablets, coated pellets of one agent incorporated into a tablet of the other, separately-coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc. Due to the incompatibility, combination products of the two agents in an injectable solution are not really acceptable. For convenience purposes, a coated compressed tablet of benazepril together with amlodipine powder in a capsule has been found to be the most desirable oral form.

If a diuretic is added it may be employed as per the below table.

TABLE 1
Diuretic Dosages

| | Typical Range | Preferred Range |
|---|---|---|
| Diuretic | (mg/day) | (mg/day |
| Bendroflumethiazide | 1.250-40 | 2.5-20 |
| Benzthiazide | 3.125-200 | 6.25-100 |
| Chlorothiazide | 62.5-2000 | 125-1000 |
| Hydrochorothiazide | 6.25-200 | 6.25-100 |
| Hydroflumethiazide | 6.25-200 | 12.5-100 |
| Polythiazide | 0.25-16 | 1-4 |
| Trichloromethiazide | 0.25-16 | 1-4 |
| Chlorthalidone | 6.25-200 | 12.5-100 |
| Indapamide | 1.25-20 | 2.5-5 |
| Metolazone | 0.25-30 | 0.5-15 |
| Quinethazone | 25-200 | 50-100 |
| Bumetanide | 0.25-40 | 0.5-20 |
| Ethacrynic Acid | 12.5-400 | 25-200 |
| Furosemide | 5-2000 | 10-200 |
| Toremide | 2.5-500 | 5-300 |
| Amiloride | 2.5-30 | 5-10 |
| Spironolactone | 12.5-400 | 25-200 |
| Triamterene | 12.5-400 | 25-200 |

For the present purposes, preferred mammals are rabbits, dogs, goats, hogs, sheep, horses, cattle and primates, more preferably primates, most preferably humans.

### EXAMPLE

The following example are presented to exemplify, but not to limit the invention.

### Example 1

### Clinical Trial

1. Rationale

The ACCOMPLISH (Avoiding Cardiovascular Events through COMbination Therapy in Patients Living with Systolic Hypertension) trial is the first major outcomes trial of initial therapy with combination antihypertensive therapy. Recognition of the importance of aggressive blood pressure control will lead to more frequent use of combination therapy. An exciting possibility is that specific drug combinations may confer target organ protection in addition to and independent of their blood pressure lowering effects. An ACE-inhibitor/diuretic combination will be increasingly used and is likely to become commonplace in the treatment of hypertension. The ACCOMPLISH study will evaluate whether Lotrel®, the combination of the ACE-inhibitor benazepril and the CCB amlodipine, provides added benefits in reducing morbidity and mortality from cardiovascular events in a high-risk hypertensive population when compared with an ACE-inhibitor (benazepril) diuretic combination (components of Lotensin HCT®).

2. Study Objectives

Primary Objective

The primary objective of this trial is to assess the time to first event of composite cardiovascular morbidity and mortality with Lotrel® compared with the combination of benazepril and hydrochlorothiazide in patients with high risk hypertension (see section 3.5.2).

Secondary Objectives

The secondary objectives of the trial are to compare composite cardiovascular morbidity, new onset diabetes, progression of renal disease and hospitalization for congestive heart failure with Lotrel® versus the combination of benazepril and hydrochlorothiazide.

Other Key Parameters

Other key parameters of the trial are to compare all-cause mortality, all hospitalizations, renal function (estimated change in glomerular filtration rate), LVH, peripheral arterial revascularization procedure or nontraumatic amputation and progression/regression of microalbuminuria (30-300 mg/g) or clinical albuminuria (>300 mg/g) with Lotrel® versus the combination of benazepril and hydrochlorothiazide.

Long term safety and tolerability of the two treatment groups will also be evaluated.

3. Investigational Plan

3.1. Overall Study Design

This will be a randomized, multicenter, double-blind, parallel-group, active-controlled study comparing the efficacy of Lotrel® to the combination of benazepril and HCTZ in high risk hypertensive patients in reducing cardiovascular outcomes. High risk patients are defined as those age ≧60 years, SBP ≧160 mmHg or currently on antihypertensive therapy, and evidence of cardiovascular disease or target organ damage (see section 3.3.2.1, Table 1). Following randomization, all patients will be treated at Dose Level 1 (see Figure 3.1) for 4 weeks, then undergo force titration to Dose Level 2 for an additional 4 weeks. Thereafter, patients will be titrated if needed to achieve a target blood pressure of <140/<90 mmHg. For patients with diabetes or chronic kidney disease, investigators are encouraged to use a target blood pressure of 130/80 mmHg. Patients are titrated to Dose Level 3 with the possibility of subsequent free add-on antihypertensive agents based on target blood pressure.

Following randomization, all patients will be treated at Dose Level 1 for 4 weeks, then force titrated to Dose Level 2 for 4 weeks. Patients with symptomatic hypotension or systolic blood pressure <100 mmHg should not be force-titrated. During the initial titration phase of the protocol, Visits 3-5 are scheduled monthly ±7 days.

Occasionally, a patient whose blood pressure was well controlled on previous antihypertensive therapy may have a rapid blood pressure rise upon the start of trial medication. Alternatively, a previously untreated patient may have a very high blood pressure. In such cases, an upward titration prior to the next scheduled visit is allowed at the investigator's discretion. Guidelines for upward titration of trial medication are: mean sitting DBP ≦110 mmHg, mean sitting SBP ≦180 mmHg, or symptoms of hypertension. In no case may a titration step be missed. Conversely, if a patient experiences symptomatic clinical hypotension on a higher dose level, they may resume treatment at the previous lower Dosage Level.

Number of Patients

A total of approximately 12,600 patients (6300 per treatment arm) that meet the study inclusion and exclusion criteria will be randomized into this study using an interactive voice response system (IVRS). The expected enrollment rate is at least 18-20 randomized patients per center.

This is an event-driven trial. Patients will be treated until the required number of randomized patients with a primary cardiovascular event (1642) is achieved. It is estimated that this study will have a total duration of approximately 5 years, including 18 months for recruitment.

3.2. Discussion of Design

ACCOMPLISH is designed to test the hypothesis that Lotrel® (amlodipine/benazepril) will reduce cardiovascular morbidity and mortality to a greater extent than a combination of benazepril/HCTZ. This study will evaluate high-risk hypertensive patients with either documented CAD, coronary equivalents (eg, diabetes) or others at high-risk for cardiovascular events. ACE-inhibitors (or ARBs) have now become the drug of choice in hypertensive patients with diabetes, renal insufficiency and/or proteinuria. Thus, the use of the ACE-inhibitor benazepril in both treatment groups allows for the inclusion of these important high-risk patient subgroups in this study.

Aggressive treatment and control of blood pressure have been shown to lower cardiovascular risk, without any clear lower threshold for blood pressure reduction. It is important that investigators attempt to achieve goal blood pressure (<140/<90 mmHg) for patients in this study; investigators are encouraged to use a lower target blood pressure goal in appropriate patients (i.e., <130/80 mmHg in patients with diabetes or chronic kidney disease).

The study provides for 2 steps of dose-titration followed by additional add-on therapy to achieve blood pressure goals. There are 3 dose levels of Lotrel® (5 mg amlodipine/20 mg benazepril, 5 mg amlodipine/40 mg benazepril, 10 mg amlodipine/40 mg benazepril) and 3 dose levels of the control (20 mg benazepril/12.5 mg HCTZ, 40 mg benazepril/12.5 mg HCTZ, 40 mg benazepril/25 mg HCTZ). In order to provide for equal and high level ACE-inhibition in both groups, patients are randomized to Lotrel® 5/20 mg or benazepril 20 mg/HCTZ 12.5 mg and force titrated to Lotrel® 5/40 mg or benazepril 40 mg/HCTZ 12.5 mg. Following the forced dose-titration, further dose-titration is based on achieving target blood pressure (<140/<90 mmHg). For those patients not reaching goal blood pressure, patients are titrated to Lotrel® 10/40 mg or benazepril 40 mg/HCTZ 25 mg, and if necessary to achieve target blood pressure, other allowed add-on antihypertensives are then used. Medication from study drug classes ACE-inhibitors, CCBs, thiazide or thiazide-like diuretics and specific inhibitors of the renin angiotensin aldosterone (RAAS) system (i.e. ARBs, aldosterone-receptor blockers) are NOT allowed as add-on therapy. Results of previous large clinical trials evaluating the effects of various monotherapies on morbidity and mortality have been confounded by the use of frequent add-on therapy, which were necessary to achieve blood pressure control. It is anticipated that a large proportion of patients in ACCOMPLISH will be controlled with randomized combination therapy, without needing further add-on therapy. This should make interpretation of study results in this regard more straightforward.

For the high-risk hypertensive group that will be enrolled in ACCOMPLISH, the annual first-event rate is assumed to be 3.5% for the control group (benazepril/HCTZ). The sample size is calculated to detect a 15% reduction in the event rate for the Lotrel® treatment group with 90% power. In order to fulfill these assumptions, a total of 1642 events at final analysis are required for both treatment groups combined. In this regard, it is crucial that efforts are made to keep patients in the trial. If a patient does permanently discontinue study treatment, the patient should nevertheless be followed for the duration of the trial.

Although not an efficacy variable, office trough blood pressure will be measured at each visit in order to evaluate blood pressure control in each of the treatment groups. To further characterize blood pressure control over 24 hours, ambulatory blood pressure monitoring (ABPM) will be performed in a subset of patients at year 2 during the study.

3.3. Study Population

3.3.1. Patient Population

The patient population will be comprised of approximately 12,600 patients with systolic hypertension ≧160 mmHg or currently on antihypertensive therapy, age ≧60 years and evidence of a cardiovascular disease or target organ damage as defined in Table 1 of section 3.3.2.1. below.

3.3.2. Inclusion and Exclusion Criteria

3.3.2.1 Inclusion Criteria

1. Men or women of any racial background
2. ≧60 years of age
3. Previously untreated or treated hypertension

Definitions:
For currently untreated Patients: mean sitting SBP readings of ≧160 mmHg at 2 consecutive readings (confirmed on two different days) during the screening period prior to randomization and mean sitting DBP readings not greater than 115 mmHg.
For patients already on antihypertensive treatment: There is no lower limit of mean sitting SBP or DBP. However, the upper limit of mean sitting SBP may not exceed 210 mm Hg or mean sitting DBP may not exceed 115 mmHg.
   Please note: Eligible patients already receiving antihypertensive treatment will be withdrawn from their antihypertensive treatment and rolled over to randomized trial medication (either Lotrel® 5/20 mg or benazepril 20 mg/HCTZ 12.5 mg) without any wash-out period. Patients at risk of rebound after withdrawal of their previous antihypertensive medication (e.g., beta-blockers or centrally acting alpha agonists) should have their medications down-titrated according to labeling directions during the Screening period, prior to being “rolled over” to study medication.

4. Evidence of at least one cardiovascular disease or target organ damage (Table 1) at Visit 1 as defined below:

TABLE 1
Cardiovascular Disease/Target Organ Damage

Prior myocardial infarction (MI).
Hospitalization for unstable angina
Coronary revascularization [coronary artery bypass graft (CABG) or
percutaneous coronary intervention (PCI)].
The above events must be appropriately documented by hospital records,
angiographic reports, ECG or other diagnostic tests.
History of stroke, verified by persistent hemiparesis, MRI or CT imaging,
angiography or appropriate hospital records.
Peripheral arterial occlusive disease documented by angiography, Doppler
studies or with previous non-traumatic leg amputation, limb bypass
surgery, percutaneous revascularization.
Diabetes defined as overnight fasting blood glucose concentration ≧ 7.0
mmol/L (≧126 mg/dL) (ADA criteria, 2 confirmatory values) or
chronic treatment with oral hypoglycemic agent and/or insulin.
Left ventricular hypertrophy (LVH) confirmed by central ECG laboratory
reading (Sokolow and Lyon criteria or Cornell criteria).
Serum creatinine defined as >1.5 mg/dL or 133 μmol/L (women) and
>1.7 mg/dL or 150 μmol/L (men)
Proteinuria defined as albumin/creatinine ratio of >300 mg/g on a spot
urine collection confirmed on 2 separate occasions.

3.3.2.2 Exclusion Criteria
1. Current angina pectoris (ie, no anginal event within 3 months prior to Visit 1)
2. Known secondary hypertension of any etiology (e.g., uncorrected renal artery stenosis).
3. Refractory hypertension defined as SBP ≧180 mmHg and/or DBP ≧110 mmHg unresponsive to triple-drug regimens of sympatholytics, diuretics and vasodilators.
4. History of symptomatic heart failure (NYHA classes II-IV) or known ejection fraction <40%.
5. Myocardial infarction, coronary revascularization (CABG or PCI), unstable angina within one month of Visit 1.
6. Stroke or transient ischemic event (TIA) within 3 months of Visit 1.
7. Current abuse or recent history of alcohol or other drug substance abuse (past 12 months).
8. Mental or legal incapacitation.
9. Participation at present or during the past 30 days in another investigational drug trial.
10. Significant obstructive valvular cardiovascular disease or any valvular disease expected to lead to surgery during the course of the study
11. Evidence of hepatic disease as determined by one of the following AST or ALT values ≧2 times the upper limit of normal.
12. Impaired renal function defined as serum creatinine ≧2.5 mg/dL (221 μmol/L)
13. Baseline serum potassium of >5.2 meq/L not on potassium supplements.
14. Gastrointestinal disorders which could interfere with drug absorption.
15. Known allergy to any of the drugs administered in the study (amlodipine, benazepril, hydrochlorothiazide)
16. History of malignancy including leukemia and lymphoma (but not basal cell skin cancer) within the last 5 years.
17. History of clinically significant auto immune disorders such as Systemic Lupus Erythematosus.
18. Significant non-cardiovascular illness or condition likely to result in death prior to trial completion, e.g., major organ transplant (life expectancy <5 years).
19. Incapacity or unwillingness to sign the informed consent.
   3.3.3. Interruption or Removal of Patients from Trial or Analysis

Every effort must be made to ensure that patients remain in the study and on study medication for the duration of the study. Each randomized patient must be followed until study completion whether or not study medication is temporarily interrupted or permanently discontinued.

3.3.3.1. Temporary Interruption of Study Medication

A temporary interruption of study medication may occasionally be required. In this case, the IVRS must also be called and the patient's discontinuation reported accordingly. If a temporary interruption occurs, study medication should be re-initiated as soon as possible. Every attempt to re-initiate study medication should be made. The re-initiation of study medication is not subject to a time limit; it may occur after days, weeks, months, or even years. The number of attempts to re-initiate is not limited.

In all cases, study medication must be interrupted for pregnancy for the duration of gestation and lactation.

When study medication is re-initiated, it is not necessary to begin again with the lowest dose level. Re-initiation of study medication may start with the previously administered dose level at the investigator's discretion.

If, at any time, the investigator determines that the patient will never be restarted on study medication, the local Novartis monitor should be contacted to discuss the rationale for this decision.

3.3.3.2. Permanent Discontinuation from Study Medication

All randomized patients, including patients with morbid endpoints, should remain on study medication until death or trial end, except when, after repeated and careful attempts to re-initiate an interrupted treatment, the following conditions exist:
Whenever the patient decides it is in his/her best interest
Whenever the investigator considers it advisable or in the patient's best interest.
Intolerable adverse experience(s).
Presence of clinically significant laboratory abnormality despite adjustment of background therapy and considered possibly attributable to study drug.

If the investigator determines that the patient is to be permanently discontinued from study medication, the local Novartis monitor should be contacted to discuss the rationale for this decision. Appropriate alternative therapy should be instituted.

ALL PATIENTS MUST BE FOLLOWED FOR THE ENTIRE DURATION OF THIS CLINICAL TRIAL. Subsequent endpoints MUST be reported for the duration of the trial (via office visits or remote contact) regardless of whether the patient is taking study medication or not. Patients may not enroll in any subsequent investigational trials until their participation on the ACCOMPLISH trial is complete.

3.3.3.3. Removal of Patients from the Trial

All efforts should be made to keep patients in the trial and compliant to the protocol required visit schedule, including patients who have permanently discontinued trial medication (as described above). If a patient refuses to return to the clinic, all methods of communication must be employed to follow the patient for clinical event evaluation (telephone, e-mail, postcard, registered letter requiring signature, fax, etc.)

A concerted effort must be made to determine the reason(s) why a patient fails to return for any protocol required visits or is discontinued from the trial. This information should be recorded in the visit specific comments section of the electronic case report form (eCRF).

If a patient discontinues from the trial for one of the following reasons, a Study Completion eCRF should be completed:
“Death”
“Withdrawn consent”
“Lost to follow-up”, i.e. not in communication with the clinic for >1 year after exhausting all means of contact

In patients who discontinue from the trial, all efforts should be made to collect the information related to the patient's primary and secondary endpoints and life status. Data collection will be limited to the source of patient information, major events, i.e. primary or secondary endpoints, and life status. Whenever possible, all available data on any endpoints reported should be compiled and sent for adjudication by the Endpoint Committee.

3.3.3.4. Removal of Patients from Analysis

There are no pre-planned reasons for removal of patients from the all-randomized-patients (intention-to-treat) analysis.

3.4. Treatments

3.4.1. Investigational Therapy and Reference Therapy

Novartis will supply the investigators with the following study medication, Lotrel® 5/20, 5/40 and 10/40 mg and benazepril 20 mg/HCTZ 12.5 mg benazepril 40 mg/HCTZ 12.5 mg and benazepril 40 mg/HCTZ 25 mg which will be sufficient for the course of the study. These medications will be supplied in bottles labeled as CIB002 as identically appearing capsules.

The medication labels will be multilingual and comply with local legal requirements. These labels will contain the medication number but will supply no information about the patient. The proper storage conditions of the study drug will also be described on the medication labels.

Randomized double-blind study medication will be supplied in packages bearing a two-part label identified by a unique medication number.

Free add-on of other antihypertensive agents will be dispensed independently from the site if target blood pressure of 140/90 mm Hg is not achieved after treatment at all 3 double-blind Dose Levels. Permitted add on medication include beta-blockers, alpha-blockers, clonidine (or other centrally acting antihypertensives) and loop diuretics.

During the double-blind treatment phases, patients will be instructed to take one capsule with water in the morning, except on the morning of their next office visit. On office visit days, study medication will be taken after completion of the visit evaluations. Patients must return medication from the previous visit before they are dispensed medication for the next visit.

3.4.2. Treatment Assignment

At Visit 1, patients meeting the inclusion/exclusion criteria will be assigned a unique patient number. The patient number will be assigned sequentially at each study center beginning with the number 1. Once assigned, a patient number will not be reused.

Medication Numbering

At randomization (Visit 2), patients will be randomized to receive the medication identified by a medication number, associated to one of the two treatment groups. The medication number is assigned to the patient via the IVRS and will appear on the medication actually dispensed to the patient. The IVRS will also assign a randomization number, which will be used by the database to link the patient to the treatment group assigned. The randomization number must be written down on the space provided on the medication label.

3.4.3. Blinding

Randomization will be performed by the IVRS vendor using a validated system that automates the random assignment of treatment group to randomization numbers. The randomization scheme will be reviewed by Novartis biostatistics quality assurance group and locked by them after approval.

Randomization data are kept strictly confidential, accessible only to authorized persons, until the time of unblinding. During the trial the IVRS will report the occurrence of any emergency code breaks immediately to the Novartis clinical trial leader (CTL) and the monitor for the site. Only when the study has been completed, the data file verified, and the protocol violations determined will the drug codes be broken and made available for data analysis.

For details of the emergency procedure for unblinding of individual patients in cases of emergency see Section 10.1.3. Emergency procedure for unblinding.

3.4.4. Concomitant Therapy

Concomitant medication may be continued throughout the study if not mentioned under Section 3.4.4.1 (disallowed concomitant medication). The patient should inform the investigator of any additional medication taken (including OTC-drugs and herbal preparation). This information should be documented in the patient's medical record at the site and not on the eCRF. Only medications listed in sections 3.4.4.1 and 3.4.4.2 will be recorded on the eCRF.

Disallowed Concomitant Treatment

At randomization, all medication previously being used to treat hypertension must be discontinued. If treatment of hypertension with any of these medications is considered mandatory, the patient should not be enrolled.

The following concomitant antihypertensive treatment may not be given throughout the whole study for any reason unless trial medication is permanently discontinued:
ACE inhibitors other than trial medication
CCB other than trial medication
Thiazide or thiazide-like diuretics (e.g., chlorthalidone, indapamide) other than trial medication
ARBs
Aldosterone receptor blockers

Patients will stay in the study even if exclusionary medication was taken.

Allowed Concomitant Treatment

The following medications may be administered to treat hypertension after completing the third titration step of the study:
Beta-blockers
Alpha-blockers
Loop diuretics
Clonidine or other centrally acting antihypertensives

We recommend first line add-on treatment of a beta-blocker (such as atenolol) unless it is felt that a diuretic is needed for volume.

The continued use of the following drugs for non-hypertension indications is allowed before and after randomization:
Alpha-blockers, e.g., for prostatic hyperplasia
Beta-blockers, e.g., for arrhythmia, secondary prevention of myocardial infarction, glaucoma
Centrally acting agents, e.g., for migraine or smoking cessation.

If a patient develops an intolerable adverse experience at the high dose level of blinded trial medication, e.g., severe intolerable edema, and causes other than a possible relationship to blinded trial medication have been ruled out, the dose of blinded trial medication may be reduced to the previous lower dose level. Free add-on of other antihypertensives may then be administered with this low dose level of blinded trial medication to control blood pressure. If the intolerable adverse experience persists, trial medication may be discontinued, but the patient must be followed per protocol for the full duration of the trial.
Do not reduce the dose level of blinded trial medication for hypokalemia or other laboratory abnormalities. For hypokalemia, potassium supplementation may be added according to local medical practice. For other laboratory abnormalities, appropriate therapy may be added as needed or trial drug discontinued if required.

Patients who develop atrial tachyarrhythmias may be treated acutely with intravenous calcium channel blockers according to local medical practice. The use of digoxin and beta blockers or any other non-calcium channel blocker antiarrhythmic may be used as chronic therapy for these patients.

3.4.5. Treatment Compliance

Records of study medication used, dosages administered, and intervals between visits will be kept during the study. Drug accountability will be noted by the field monitor during site visits and at the completion of the trial. Patients will be asked to return all unused medication at the end of the study.

3.5. Visit Schedule and Assessments

3.5.1. Visit Schedule

During this trial at least 11 visits will be performed in a period of 3-5 years. Each evaluation will be conducted in the morning, allowing a range of +/−7 days for scheduling purposes for Visits 3-5 and +/−28 days for Visits 6-11. Additional interim visits may be conducted as needed to ensure blood pressure control.

Please note that this flexible visit schedule is proposed as a GUIDE and specific circumstances should be discussed with the study monitor. Visits falling outside this window SHOULD NOT LEAD to patient discontinuation. Instead, patients should be seen as soon as possible and subsequent visits should be scheduled in line with Visit 2 (Day 1). Patients should be instructed NOT to take study medication on the morning prior to their office visit. If the patient took trial medication prior to arriving for their scheduled visit, the visit must be postponed and rescheduled to ensure a trough blood pressure evaluation. Please note that if circumstance exists where the study patient is unable to be attend morning clinics (i.e. evening shift worker, etc.), afternoon evaluations are permitted but must occur ˜24 hours after the last dose of study medication, must always take study medication in the afternoon and must be able to present for lab evaluations in a fasted state when necessary.

TABLE 3-1
Evaluation and visit schedule

| | Visit³ | | | | | | |
|---|---|---|---|---|---|---|---|
| | | | | | | | 7-11 |
| | 1¹ | 2 | 3 | 4 | 5 | 6 | Years |
| | Week-2 | Day 1 | Month 1 | Month 2 | Month 3 | Month 6 | 1-3² |
| Inclusion/Exclusion Criteria | X | | | | | | |
| Medical History/Background | X | | | | | | |
| Information | | | | | | | |
| Physical Examination - Complete | X | | | | | | |
| Physical Examination - Interim | | X | | | X | X | X⁴ |
| Blood Pressure and Pulse | X | X | X | X | X | X | X |
| Ambulatory Blood Pressure | | | | | | | X |
| Monitoring⁵ | | | | | | | |
| ECG⁶ | *⁷ | X | | | | | X⁸ |
| Laboratory Evaluations⁹ | | | | | | | |
| Hematology | X | | | | | | X¹⁰ |
| Chemistry | LC | | SC | | SC | | SC/LC¹¹ |
| Urine albumin/creatinine | X¹² | | | | | | X¹² |
| ratio | | | | | | | |
| Biomarker Evaluation | X | | | | | | X¹³ |
| Pharmacogenetic Sample¹⁴ | | | X | | | | |
| Screening Log¹⁵ | X | | | | | | |
| Randomization | | X | | | | | |
| IVRS call | | X | X | X | X | X | X |
| Dispense Study Medications | | X | X | X | X | X | X |
| Drug Accountability | | | X | X | X | X | X |
| Dose Titration Evaluation¹⁶ | | | X | X | X | X | X |
| Occurrence of Clinical Endpoints | | | X | X | X | X | X |
| Prior/Concomitant Medications | X | X | X | X | X | X | X |
| Adverse Events/SAEs | X | X | X | X | X | X | X |
| Final Study Visit¹⁷ | | | | | | | X |

¹Washout-Patients will discontinue taking anti-hypertensive medications, according to manufacturer's recommendations at Visit 1.
²Evaluations will be conducted at least every 6 months from Visit 6-11. If the study duration is longer than the 3 years presented in Table 3-1, patients will continue to be evaluated every 6 months until the study is complete as required. At the final study visit, evaluations listed for Visits 7-11 will be followed.
³+/−7 days during Visits 3-5 and +/−28 days during Visits 6-11. (always return patients to original visit schedule calculated from Visit 2/Day 1).
⁴Interim physical exams to be done yearly.
⁵To be performed in 560 patients at specified study sites at year 2.
⁶Submitted to the Central ECG lab (See Section 3.5.4).
⁷To be completed at Visit 1 for patients whose ONLY qualifying inclusion criterion is LVH. Patients included in trial based on all other CV Table 1 criteria (Section 3.3.2.1.) will have ECG done at Visit 2.
⁸Done at Visit 8 (18 mo) and Visit 11 (year 3);
⁹Blood will be drawn in a fasted state (hematology and blood chemistry).
¹⁰Done at Visits 8 (18 mo) and Final Study Visit.
¹¹SC (short chemistry) to be done yearly; LC (long chemistry) to be completed at Visit 8 (18 mo) and at the final study visit.
¹²spot urine for albumin/creatinine ratio at Visit 1, repeat prior to Visit 2 if patient's only qualifying criterion is proteinuria (>300 mg/gm, Section 3.3.2.1.), if microalbuminuria (≧30 mg/gm) present at Visit 1 then yearly urinalysis required (see Section 3.5.4.).
¹³Done at Visit 7 (1 yr) and Final Study Visit.
¹⁴Participating patients must sign a separate pharmacogenetic specific consent form.
¹⁵To be completed for patients not entering the double-blind treatment phase. To be maintained at the investigational site.
¹⁶Visit 3: Forced titration; Visit 4 and subsequent visits: Upward titration of study drugs to achieve target blood pressure. Dose titration may occur at any visit patient does not achieve target blood pressure.
¹⁷Follow-up Evaluation Form to be completed for patients who do not report to clinic for study visit.

3.5.2. Efficacy Assessments

All clinical endpoints and fatal events will be processed by completing an Assessment of Endpoint Form and forwarding all relevant information to the Endpoint Committee for adjudication.

3.5.2.1. Primary Endpoint

Time to first event of composite cardiovascular morbidity and mortality:

Cardiovascular Morbidity, defined as
Non-fatal, clinically-evident acute myocardial infarction.
Non-fatal stroke
Hospitalization for unstable angina
Coronary revascularization procedure (PCI or CABG)

Cardiovascular Mortality, defined as death due to:
Sudden cardiac death, fatal MI, fatal stroke, death due to coronary intervention, death due to CHF or other CV causes.
   3.5.2.2. Secondary Endpoints

The secondary endpoints will be assessed separately as follows:
Composite cardiovascular morbidity
New onset diabetes (ADA definition)
Progression of renal disease as defined by a doubling of serum creatinine or progression to end-stage renal disease.
Hospitalization for congestive heart failure
   3.5.2.3. Other Endpoints
1. All-cause mortality
2. All hospitalizations
3. Renal function (estimated change in glomerular filtration rate).
4. LVH by ECG.
5. Peripheral arterial revascularization procedure or nontraumatic amputation.
6. Progression/regression of microalbuminuria (30-300 mg/g) or clinical albuminuria (>300 mg/g)

Subgroups of patients with 1) diabetes at baseline, 2) coronary artery disease at baseline, 3) chronic renal insufficiency at baseline (ie, serum creatinine >1.5 mg/dL (women) and >1.7 mg/dL (men) will be evaluated. Subgroups will also be evaluated by gender, race and age (<70, ≧70 yrs).

3.5.3. Procedure Description

Blood Pressure Measurement

During screening prior to randomization blood pressure will be recorded. The arm in which the highest sitting diastolic pressures are found will be the arm used for all subsequent readings throughout the study. All attempts should be made to have the same individual obtain blood pressure readings in each individual patient at each visit at the same time of the day with the same equipment.

Using a calibrated standard sphygmomanometer or a calibrated digital device and appropriate size cuff, arterial blood pressure determinations will be made in accordance with the 1988 AHA Committee Report on blood pressure determination (Hypertension 11: 210A-222A, 1988). With the arm supported at the level of the heart, systolic pressure will be recorded when the initial sound is heard (Phase I of the Korotkoff sound); diastolic pressure will be recorded at the disappearance of sound (Phase V of the Korotkoff sound). At each study visit, after having the patient in a sitting position for five minutes, systolic/diastolic blood pressure and heart rate will be measured three times. The repeat measurements are to be made at one to two-minute intervals. The cuff should be deflated at a rate not greater than 2 mmHg/sec.

Pulse Rate

At each visit, the pulse rate will be measured for 30 seconds just prior to the seated blood pressure measurements.

Ambulatory Blood Pressure Monitoring

ABPM will be conducted during the study at Year 2 in approximately 560 patients at selected sites. The ambulatory blood pressure monitor will be placed on the non-dominant arm.

ABPM Readings, Quality Control Criteria

The ABPM unit will be automatically set to measure and record blood pressure based upon study specific requirements. The inflation sequence for this protocol will be outlined in the ABPM Training Manual. At the completion of each 24-hour ABPM period, each ABPM report will be immediately evaluated against a set of quality control criteria designed for this study. If the quality control criteria are not met, the patient will be requested to repeat the 24 hour monitoring period. See ABPM Study Training Manual for further details.

The following procedures will be done over two days at Year 2 Visit:

Application

The patient will appear to clinic for the Year 2 Study Visit. The office blood pressure reading will be done, along with all other study evaluations. The ABPM device will be applied and be determined to be operating appropriately. The patient will then have his/her dose of double blind study medication administered and the time of administration recorded.

Removal

The patient will be instructed to return the next day approximately 24 hours later for removal of the device. The device will be removed, (≧24 hours since last dose of medication) and determined that the readings met quality control criteria. If the readings did not meet quality control, the patient will be asked to continue taking double blind medication and repeat the ABPM within 3 days.

The procedure should be repeated (within 1 month) if the reading is not acceptable.

3.5.4. Safety Assessments

Safety assessments will consist of monitoring and recording the pre-defined safety and tolerability endpoints (see below), all serious adverse events, the regular monitoring of hematology and blood chemistry, regular measurement of vital signs and the performance of physical examinations. ECG evaluation will also be performed. Results of all safety assessments should be maintained in the patients study chart (source documents).

Pre-Defined Safety and Tolerability Parameters

The following pre-defined safety and tolerability endpoints are known side effects of either Lotrel® or benazepril and hydrochlorothiazide:
Cough
Dizziness
Peripheral edema
Angioedema
Allergic reaction to study drugs
Hypo/Hyperkalemia (these parameters will be identified by the central lab and will not be listed on the adverse event eCRF).

Information on the occurrence of these adverse events will be collected and recorded on the eCRF for all patients.

Adverse Events

Adverse events will be recorded in the eCRF or the Serious Adverse Event (SAE) form if they meet the following criteria:
Primary and secondary efficacy parameters (as described in Section 3.5.2)
Pre-specified safety and tolerability parameters (known side effects of either Lotrel, benazepril and/or hydrochlorothiazide) as described in the previous section above.
Serious adverse events (as described in the following section)

As far as possible, each adverse event will also be described by:
1. its duration (start and end dates),
2. the severity grade (mild, moderate, severe)
3. its relationship to the study drug (suspected/not suspected),
4. the action(s) taken and, as relevant, the outcome.

Other non-serious adverse events will not be collected in the eCRF. However, information about all adverse events, whether volunteered by the patients, discovered by investigator questioning, or detected through physical examination, laboratory test or other means, will be recorded in the patient's study chart (source documents) and the events will be followed and treated as appropriate. An adverse event is any undesirable sign, symptom or medical condition occurring after starting study treatment, even if the event is not considered to be treatment-related. Medical conditions/diseases present before starting study treatment are considered adverse events only if they worsen after starting study treatment. Abnormal laboratory values or test results constitute adverse events only if they induce clinical signs or symptoms or require therapy or a change in therapy.

Serious Adverse Events

A serious adverse event is an undesirable sign, symptom or medical condition which:
1. is fatal or life-threatening
2. required or prolonged hospitalization
3. results in persistent or significant disability/incapacity
4. constitutes a congenital anomaly or a birth defect
5. is medically significant, in that it may jeopardize the subject and may require medical or surgical intervention to prevent one of the outcomes listed above.

Events not considered to be serious adverse events are hospitalizations for the:
routine treatment or monitoring of the studied indication, not associated with any deterioration in condition.
treatment, which was elective or pre-planned, for a pre-existing condition that is unrelated to the indication under study and did not worsen
admission to a hospital or other institution for general care, not associated with any deterioration in condition
treatment on an emergency, outpatient basis for an event not fulfilling any of the definitions of serious given above and not resulting in hospital admission.

Any serious adverse event occurring after the patient has provided informed consent and until 4 weeks after the patient has stopped study participation must be reported. This includes the period in which the study protocol interferes with the standard medical treatment given to a patient (e.g. treatment withdrawal during washout period, change in treatment to a fixed dose of concomitant medication).

Serious adverse events occurring more than 4 weeks after study discontinuation need only be reported if a relationship to the Novartis study drug (or therapy) is suspected.

To ensure patient safety each serious adverse event suspected by the investigator to be related to study medication must be reported to Novartis within 24 hours of learning of its occurrence.

Serious adverse events not suspected by the investigator to be related to study medication will be reported to the Novartis with the eCRF and/or endpoint documentation. Instructions about completing initial and follow-up Serious Adverse Event Report Forms and sending them to Novartis are given in Section 10.1.1. Instructions for rapid notification of serious adverse events.

Laboratory Evaluations

Blood samples will be obtained in a fasted state (8 hours without food or beverage).

Laboratory tests to be performed by the central laboratory as outlined below:
Hematology: hemoglobin, hematocrit, red blood cell count, white blood cell count, platelet count to be done at Visits 1 (Week −2), 8 (Month 18) and Final Study visit.
Biochemistry: Long Chemistry(LC):
   AST, ALT, alkaline phosphatase, bilirubin, creatinine, uric acid, sodium, potassium, fasted plasma glucose, total cholesterol, HDL-C, albumin, BUN or urea will be done at Visits 1 (Week −2), 8 (Month 18) and Final Study visit.

Short Chemistry (SC):
Creatinine, sodium, potassium, fasted plasma glucose will be obtained at Visits 3 (month 1), 5 (Month 3), and yearly beginning at Visit 7 (year 1).
Spot Urine: Spot urine for albumin/creatinine ratio at Visit 1; repeat prior to Visit 2 if patient's only qualifying inclusion criterion is proteinuria (>300 mg/gm, Section 3.3.2.1.). If microalbuminuria (≧30 mg/gm) is present at Visit 1, then yearly spot urine evaluation is required (see Section 3.5.4.).

All safety results will be communicated to the investigators and the sponsor. Details on the collection, shipment of samples and reporting of results by the central laboratory are provided to investigators in the laboratory manual.

Laboratory abnormalities that exceed the boundaries of a clinically notable abnormality (see Section 8.1) should be commented on by the investigator on the Comment eCRF page and additional evaluations should be performed, as judged appropriate. If the laboratory abnormality is the primary reason for an unforeseen hospitalization or fulfills otherwise the seriousness category of an Adverse Event, the procedure for rapid notification of serious adverse events must be followed. Likewise, if the laboratory abnormality leads to discontinuation, the patient should be followed until the abnormality resolves or is judged to be permanent.

Vital Signs

Body weight (with the patient in street clothes and without shoes) will be measured yearly and vital signs (pulse and BP) will be measured at Visits 1-5 and every 6 months from Visits 6-11. If the study duration is longer than the 3 years presented in Table 3-1, patients will continue to be evaluated every 6 months until the study is complete.

If any abnormal vital signs are present, the patient should be monitored at least hourly until normal values are obtained or the abnormality can be satisfactorily explained. A comment is required if any vital signs fulfill notable criteria.

Physical Examination

For all physical examinations attention should be focused on cardiovascular signs and symptoms. The extensive physical examination (Visit 1) comprises the examination of head, thorax, abdomen, spinal column and the measurements of body weight, body height, auscultation of heart, lungs and abdomen, and the inspection of skin. The interim physical examination comprises a short check of all organ systems including auscultation of heart and lungs and a check for signs and symptoms of cardiovascular diseases and will be done at Visits 2 (Day 1), 5 (Month 3), 6 (Month 6), 7 (Year 1), 9 (Year 2), 11 (Year 3) and yearly through study completion and at the Final Study Visit.

Information about the physical examination must be present in the source documentation at the study site.

ECG

A 12-lead ECG will be performed at Visit 1 (Day 1), Visit 8 (Month 18) and at Visit 11 (Year 3). All ECGs must be identified with screening number/patient number, patient initials, and date/time of recording. All ECGs will be sent for central ECG reading. The following ECG variables will be recorded at the central reading: cardiac rhythm, myocardial infarction and LVH (Sokolow Lyon or Cornell criteria).

Note that if a patient must qualify on the LVH inclusion criterion (ie, patient does not have any of the other CV disease/target organ damage listed in Table 1), the ECG should be performed at the screening visit (visit 1) rather than at visit 2. This ECG must be faxed for central reading for LVH verification (yes/no) prior to randomization.

Biomarker Assessment

High sensitivity C-reactive protein (hs-CRP) and other predictors of cardiovascular disease will be measured at Visits 1, 7 and Final Study Visit. The parameters to be measured would be restricted to those that would lead to a better understanding of the pathophysiology of cardiovascular disease, prediction of the development of the condition or responses to treatment. For these measurements plasma and serum aliquots will be frozen until the time of analysis.

3.5.5. Drug Levels and Pharmacokinetic Assessments

None planned

3.5.6. Pharmacogenetic Assessments

To study the effects of human genetic variation on drug response, we plan to conduct exploratory pharmacogenetics research studies as a sub-study to this protocol. See Post Text Supplement 1 for details.

3.5.7. Resource Utilization Assessment

In-patient hospitalizations is the only resource utilization parameter to be followed during the study.

4. Protocol Amendments, Other Changes in Study Conduct

4.1. Protocol Amendments

Any changes or addition to this protocol requires a written protocol amendment and this must be approved by Novartis, the investigator, and the Institutional Review Board (IRB) before the change or addition can be considered effective.

4.2. Other Changes in Study Conduct

Changes in the conduct are not permitted. Any unforeseen changes in study conduct will be recorded in the clinical study report.

5. Data Management

5.1. Data Collection

The Investigator or designated staff must enter the information required by the protocol into the Novartis eCRF using a Novartis-supplied computer loaded with fully validated software that conforms to FDA requirements for electronic data capture. During system failures data is captured on paper Case Report Forms and is later transferred to electronic Case Report Forms.

Automatic validation programs check for data discrepancies in the electronic Case Report Form and, by generating appropriate error messages, allow modification or verification of the entered data before transfer to Novartis via a secure internet link.

The Investigator must certify that the data are complete and accurate by applying an electronic signature to the electronic Case Report Form and later receives a CD-ROM or paper copies of the patient data for archiving at the investigational site. All electronic Case Report Forms sent to Novartis by investigational sites are reviewed upon receipt for any serious adverse events.

5.2. Database Management and Quality Control

Data items are entered directly into the study database or indirectly from source data documents by designated Novartis-trained investigator staff using single data entry with electronic verification. Novartis staff review the data for completeness and accuracy and instruct the site personnel to make any required corrections or additions. Queries are generally sent to the investigational site using an electronic data query system which provides an automatic audit trail of the corrections made by designated investigator staff. Occasionally, when queries are sent on a Data Query Form, the signed, original and resolved Data Query Form is kept at the investigator site and a copy sent to Novartis so the resolutions can be entered centrally into the database.

Concomitant medications entered into the database will be coded using the WHO Drug Reference List which employs the Anatomical Therapeutic Chemical classification system. Coexistent diseases and adverse events will be coded using the Medical dictionary for regulatory activities (MedDRA) terminology.

Laboratory samples will be processed centrally through Medical Research Laboratories Incorporated (MRLI) and the results will be sent electronically to Novartis.

When the database has been declared to be complete and accurate, the database will be locked. Any changes to the database after that time can only be made by joint written agreement between the Clinical Trial Leader, the Trial Statistician and the Data Manager.

6. Statistical Methods

6.1. Statistical Methods

This is an event-driven trial. The primary objective of this trial is to evaluate the efficacy of Lotrel® (amlodipine/benazepril) compared with the combination of benazepril and hydrochlorothiazide in high risk hypertensive patients on the incidence of composite cardiovascular morbidity and mortality. The primary treatment comparison for the assessment of this primary objective will be made using a log-rank test. The secondary objectives of the trial are to examine the effects of the two comparators on the following secondary endpoints: (1) composite cardiovascular morbidity (2) new onset diabetes (3) progression of renal disease as defined by doubling of serum creatinine or progression to end-stage renal disease, and (4) hospitalization for congestive heart failure.

The primary null hypothesis tested is that the risk ratio (hazard ratio) between the two treatment groups is equal to 1 versus the alternative hypothesis that the risk ratio is not equal to 1 for the primary composite endpoint. The point estimate and confidence interval for the risk ratio between the two treatment groups will be provided using an univariate Cox regression analysis which includes only treatment in the model.

For the four secondary endpoints, to preserve type I error rate in the multiple tests, the significance testing will be carried out by Hochberg procedure.

It is planned that the data from all centers that participate in this protocol will be combined, so that an adequate number of patients will be available for analysis.

Data will be summarized with respect to demographic and baseline characteristics, efficacy observations and measurements, and safety observations and measurements.

6.1.1. Populations

Intent-to-Treat (ITT) Population

The intent-to-treat (ITT) population consists of all randomized patients. The primary analysis will be performed on the basis of ITT population.

Per-Protocol (PP) Population:

The per-protocol population consists of all randomized patients without a major protocol violation which would be considered to significantly impact efficacy assessments. Criteria for determining protocol violations used to identify patients in the per-protocol population will be defined prior to unblinding treatment codes for analysis.

A supplementary analysis of data from the per-protocol population is planned for the primary efficacy endpoint, time to first event of composite cardiovascular morbidity and mortality, using the censoring times described below, as appropriate. This supplementary analysis will be compared with the primary analysis of ITT population to examine any critical effect due to major protocol violations. The criteria for designation of patients as “per-protocol” will be determined prior to unblinding treatment codes for analysis.

Safety (SAF) Population

All randomized patients who take at least one dose of study medication and have at least one post-baseline safety assessment.

6.1.2. Background and Demographic Characteristics

Comparability between treatment groups will be examined based on the ITT population for demographic, medical history, and baseline efficacy variables.

Treatment comparability will be examined for the following variables using Chi-square test:
Age (<70, ≧70)
Sex
Race (White, Black, and Other)
Previous antihypertensive treatment (yes/no)
Diabetes (yes/no)
Evidence of at least 1 coronary heart disease (CHD) (yes/no)
   Myocardial infarction (MI)>1 month prior to Visit 1
   Coronary revascularization (CABG, PCI)>1 month prior to Visit 1
   Hospitalization for unstable angina >1 month prior to Visit 1
   History of Stroke >1 month prior to Visit 1
Evidence of at least 1 target organ damage (yes/no)
   Left ventricular hypertrophy (LVH) (confirmed at Visit 1)
   History of or present peripheral arterial disease (verified at Visit 1)
   Proteinuria (confirmed at Visit 1)
   Chronic renal insufficiency defined as serum creatinine >1.5 mg/dL or 133 mmol/L for women, and >1.7 mg/dL or 150 umol/L for men (central laboratory result at Visit 1)
Evidence of meeting individual inclusion criterion for cardiovascular disease and target organ damage listed in Table 1 under Section 3.3.2.1

Comparability between treatment groups for the ITT population will be examined using two-sample t-test for the baseline values of the following variables:
Age
Height
Weight (At Visit 1)

In addition, treatment group comparability for baseline (pre-randomization) mean sitting systolic blood pressure (BP), mean sitting diastolic BP, serum glucose concentration, serum creatinine concentration, and lipid profile (total cholesterol and HDL-C) will be examined using two-sample t-test.

All tests of baseline comparability will be based on a null hypothesis of no treatment difference and will be made at the two-sided 5% (0.05) significance level. However, these p-values are provided for descriptive purposes, and are not to be considered to define any formal basis for determining factors that should be included in statistical models. If imbalance of treatment group with respect to some variables do occur, supplemental analyses of covariance with addition of these variables may be performed to assess the impact for efficacy as appropriate.

6.1.3. Study Medication

Duration and numbers of patients on study medication will be summarized by treatment group, dose level and visit. Frequencies and percentages of patients will also be provided for maximum dose and final dose taken.

6.1.4. Concomitant Therapy

Concomitant use of antihypertensive agents during the trial will be summarized by treatment group and medication class (e.g., beta blockers, alpha blockers, loop diuretics).

6.1.5. Efficacy Evaluation

Primary Endpoint

Time to first event of composite cardiovascular morbidity and mortality, with:

Cardiovascular Morbidity, defined as

Non-fatal, clinically-evident acute myocardial infarction.
Non-fatal stroke
Hospitalization for unstable angina
Coronary revascularization procedure (PCI or CABG)

Cardiovascular Mortality, defined as death due to:

Sudden cardiac death, fatal MI, fatal stroke, death due to coronary intervention, death due to CHF or other CV causes.

Secondary Endpoints

The secondary endpoints include the following four individual time-to-event variables:
Composite cardiovascular morbidity, defined as nonfatal, clinically-evident acute myocardial infarction; nonfatal stroke; hospitalization for unstable angina; and coronary revascularization procedure (PCI or CABG)
New onset diabetes
Progression of renal disease as defined by a doubling of serum creatinine or progression to end-stage renal disease
Hospitalization for congestive heart failure
   Other Endpoints

Other endpoints of interest include:
All cause mortality
All hospitalizations
Renal function (estimated change in glomerular filtration rate (GFR)). Where, GFR will be calculated at baseline and at endpoint by Abbreviated MDRD (Modification of Diet in Renal Disease) Study Equation³⁵ displayed in below:

$$Estimated GFR ( ml \text{/} min \text{/} 1.73 {m}^{2} ) = 186 \times {( serum creatinine in mg \text{/} dl )}^{- 1.154} \times {( Age in years )}^{- 0.203} \times ( 0.742 if female ) \times ( 1.210 if African \text{-} American )$$
LVH by ECG
Peripheral arterial revascularization procedure or nontraumatic amputation.
Progression/regression of microalbuminuria (30-300 mg/g) or clinical albuminuria (>300 mg/g)

In general, all of the efficacy endpoints can be classified into two categories: (1) time-to-event variables, and (2) non-time-to-event variables.

Time-to-Event Variables

For time-to-event efficacy variables, the time to event for a given patient will be calculated as the difference between the patient's date of event (during the double-blind period) and the date of randomization. For patients with multiple occurrences of a given endpoint, the time to first occurrence will be used in the analysis.

The primary dataset analyzed for all time-to-event variables will be based on ITT population. The trial will be completed when the required 1642 randomized patients with a primary event are observed or when statistically significant interim analysis results are obtained.

For patients with an observed endpoint, endpoints occurring at or prior to trial completion (or analysis cut-off date if earlier) will be included in the primary analysis as non-censored events whether those endpoints occur before or after permanent discontinuation of double-blind medication. For all events, the time from randomization date to event date will be used as the non-censoring time in the primary analysis.

A supplementary analysis will be also performed in addition to the primary analysis. In the supplementary analysis, only events which occur prior to or equal to 30 days after the time of permanent discontinuation of double-blind medication will be treated as non-censored events, and the time from randomization date to event date will be used as the non-censoring time in the supplementary analysis. However, events that occur more than 30 days after permanent discontinuation of double-blind medication will be considered as censored and the time from randomization to the 30^{th} date after permanent discontinuation of double-blind medication will be used as the censoring time in the supplementary analysis. The results obtained from the supplementary analysis will be compared with those obtained from the primary analysis to assess the effect of permanent discontinuation of double-blind medication.

For patients remaining in the trial until trial completion, with no endpoint occurring prior to trial completion (or analysis cut-off date if earlier), the time to endpoint will be considered censored and the time from randomization to trial completion (or analysis cut-off date) will be used as the censoring time in the primary analysis.

For patients who discontinue from the trial (e.g., due to withdrawal of consent, or loss to follow-up) with no events observed, the time to event will be considered censored and the time from randomization to the date the patient was considered discontinued from the trial will be used as the censoring time in the analysis.

The number of patients who discontinue from the trial is expected to be small, since even patients who permanently discontinue trial treatment are expected to remain in the trial with follow-up until trial end. Events occurring after interim analysis cut-off dates will be considered censored for the purposes of the corresponding interim analysis.

In the supplementary analysis, for those patients who have no endpoint and permanently discontinue double-blind medication during the trial, the censoring time will be the time from randomization to the 30^{th} date after permanent discontinuation of double-blind medication.

Non-Time-to-Event Variables

Non-time-to-event variables will be analyzed at each scheduled measurement time point, and will be based on the ITT population for a given variable. Although efforts will be made to collect measurements per design, missing values may still occur and will be replaced by the last observation carried forward approach. This procedure will be applied independently to the post-baseline values of each variable analyzed.

Analyses

Primary Efficacy Endpoint

For the primary endpoint, time to first event of combined cardiovascular morbidity and mortality, the primary analysis will be based on treatment comparison for the ITT population using a log-rank test. This test will be performed at an overall two-sided significance level of 0.05, using an O'Brien-Fleming boundary and a Lan-DeMets alpha-spending function for the interim analyses discussed in Section 6.1.7. The superiority efficacy of Lotrel® can be concluded if a positive risk reduction in favor of Lotrel® is statistically significant. The point estimate for the risk ratio and corresponding confidence interval (CI) will be obtained from an univariate Cox regression which includes only treatment in the model.

As an exploratory analysis for the primary endpoint, a Cox regression model with an adjustment for mean sitting systolic blood pressure as a time-dependent covariate (in addition to treatment effect) will be used to assess whether or not the treatment effect on outcome is fully mediated through blood pressure. Additionally, diastolic blood pressure may be considered as a time-dependent covariate as appropriate.

Secondary Efficacy Endpoints

The log-rank test and the univariate Cox regression model (with treatment effect only) described above for the primary analysis of the primary efficacy endpoint will be used for each of the four secondary time-to-event endpoints (i.e., composite cardiovascular morbidity, new onset diabetes, progression of renal disease as defined by a doubling of serum creatinine or progression to end-stage renal disease, and hospitalization for congestive heart failure). The point estimate and the corresponding 95% confidence interval for the risk ratio will be reported based on the univariate Cox regression analysis including only treatment effect in the model. For these four secondary endpoints, to preserve an overall type I error rate of 0.05 in the multiple tests, the significance testing will be carried out using Hochberg procedure.

For the analyses described in below (i.e. for other efficacy endpoints and 24-hour ambulatory blood pressure), treatment comparison will be based on a null hypothesis of no treatment difference versus a two-sided alternative hypothesis that there is a treatment difference. All analyses will be carried out at a two-sided significance level of 5% (0.05) and the corresponding 95% confidence interval will be provided.

Other Efficacy Endpoints

For each of the five dichotomous efficacy endpoints (i.e. all cause mortality, all hospitalizations, LVH by ECG, peripheral arterial revascularization procedure or nontraumatic amputation, and progression/regression of microalbuminuria or clinical albuminuria), the event rates will be compared between the two treatment groups by Chi-square test as appropriate. If the event times are available, these dichotomous variables will be analyzed by the same type of time-to-event analyses as described in the above for the primary and secondary efficacy endpoints (i.e. log-rank test and the univariate Cox regression model).

For the remaining non-time-to-event, continuous endpoint: renal function (estimated change in GFR), a two-way analysis of covariance (ANCOVA) will be performed at trial completion with change from baseline of estimated GFR as the dependent variable, treatment and center as two factors and baseline GFR as a covariate. The treatment comparison with its 95% confidence interval will be made based on this model. A supplementary ANCOVA model with treatment-by-center and treatment-by-baseline interaction will also be performed to assess these treatment interaction terms.

24-Hour Ambulatory Blood Pressure

Hourly Mean Ambulatory Systolic Blood Pressure (MASBP)

The hourly mean ambulatory systolic blood pressure will be calculated for each post-dosing hour over 24 hours by taking the average of the readings taken in the corresponding post-dosing hour at Year 2 visit. To assess the treatment effect on hourly MASBP (hour 1, 2, 3, . . . , 24), the analysis of covariance (ANCOVA) for repeated measures will be employed. The office systolic blood pressure measurement at baseline will be the covariate in the ANCOVA model. The factors in the ANCOVA model will include: treatment, center, post-dosing hours (hour 1, 2, 3, . . . , or 24), treatment by post-dosing hours interaction, and subject as the repeated cluster variable. Appropriate contrasts will be used to assess the treatment difference for 24-hour mean as well as for each post-dosing hourly means.
Hourly Mean Ambulatory Diastolic Blood Pressure (MADBP)

The same analysis as hourly MASBP will be carried out for hourly MADBP.
Mean Ambulatory Systolic Blood Pressure (MASBP) for Daytime/Nighttime

The analysis of covariance (ANCOVA) for repeated measures will be used to assess the treatment effect on daytime/nighttime MASBP at Year 2 visit. Each patient's daytime mean will be the average of all readings taken between 6 a.m. and 10 p.m. (>6 a.m. and ≦10 p.m.). Whereas the nighttime mean will be the average of all readings taken between 10 p.m. and 6 a.m. (>10 p.m. and ≦6 a.m.). The office systolic blood pressure measurement at baseline will be the covariate in the ANCOVA model. The factors in the ANCOVA model will include: treatment, center, time (daytime, nighttime), treatment by time interaction, and subject as the repeated cluster variable. Appropriate contrasts will be used to assess treatment differences for daytime mean as well as for nighttime mean.
Mean Ambulatory Diastolic Blood Pressure (MADBP) for Daytime/Nighttime

The same analysis as MASBP for daytime/nighttime will be carried out for daytime/nighttime MADBP.

Assessment of Potential Prognostic Covariates

To investigate potentially important prognostic covariates, supplementary analyses for the primary variable will be made using Cox regression analysis. In addition to treatment effect, the following potential covariates will be considered: baseline mean sitting diastolic blood pressure (BP), baseline mean sitting systolic BP, baseline pulse pressure (difference in baseline mean sitting systolic and diastolic BP (systolic-diastolic)), baseline serum glucose, baseline serum creatinine, baseline lipid profile (i.e. total cholesterol and HDL), and other covariates identified prior to final database lock.

Subpopulations

Patients with and without the following characteristics will be evaluated 1) diabetes at baseline, 2) coronary artery disease at baseline, 3) chronic renal insufficiency at baseline (i.e., serum creatinine >1.5 mg/dL (women) and >1.7 mg/dL (men), and 4) previous antihypertensive treatment

The purpose of these analyses is to assess the efficacy of Lotrel® (amlodipine/benazepril) compared with the combination of benazepril and hydrochlorothiazide within each individual subpopulation. The analyses for each subpopulation will be performed at trial completion for the primary efficacy endpoint (combined cardiovascular morbidity and mortality), as well as for secondary and other efficacy endpoints. Similarly, the log-rank test and Cox regression model described above for the time-to-event variables will be used for the subpopulation analyses. Treatment comparison for each subpopulation analysis will be based on the null and alternative hypotheses of no and some treatment difference, respectively, and corresponding tests will be made at a two-sided 0.05 significance level. Ninety-five percent confidence intervals for the risk ratio will also be provided.

In addition, subpopulation analyses for gender, race (African-American vs. non-African-American), and age groups (<70 vs. ≧70) will also be explored.

6.1.6. Safety Evaluation

The assessment of safety will be based mainly on the frequency of adverse events and on the number of laboratory values that fall outside of pre-determined ranges. Other safety data (e.g. electrocardiogram, vital signs, special tests) will be considered as appropriate.

Adverse events will be summarized by presenting, for each treatment group, the number and percentage of patients having any adverse event, having an adverse event in each body system and having each individual adverse event. Any other information collected (e.g. severity or relationship to study medication) will be listed as appropriate.

Laboratory data will be summarized by presenting numbers and percentages of patients outside of the extended normal ranges, by presenting summary statistics of raw data and change from baseline values (means, medians, standard deviations, ranges) and by the flagging of notable values in data listings.

Data from other tests (e.g. electrocardiogram or vital signs) will be listed, notable values will be flagged, and any other information collected will be listed as appropriate. Any statistical tests performed to explore the data will be used only to highlight any interesting comparisons that may warrant further consideration.

6.1.7. Interim Analyses

Regularly scheduled interim analyses and one final analysis are planned for the primary efficacy endpoint. The exact timing and frequency of the interim analyses will be determined and planned by an independent Data Monitoring Committee (DMC).

The a priori stopping rule to asses efficacy for early termination of this trial will be based on an O'Brien-Fleming boundary using a Lan-DeMets alpha-spending function, with a two-sided significance testing for the primary efficacy endpoint. If statistically significant risk reduction in favor of Lotrel® treatment is observed based on the specified boundary, the trial may be terminated early for conclusion of a favorable benefit. The detailed plan and procedures for interim monitoring of efficacy and safety will be provided by the independent DMC in DMC charter.

Cutoff dates for these interim analyses will be determined prior to the release of databases and treatment codes to the independent personnel performing the interim analyses (see below). For each interim analysis, the data set analyzed will consist of all patients randomized prior to the cutoff date.

The interim analyses will be performed by an independent Novartis statistician who will not be involved in the conduct of the trial. The interim analysis results will be sent directly to the independent DMC. All investigators and Novartis employees involved in the conduct and/or monitoring of the trial or in the analysis of the final trial results will remain blinded to treatment codes and to the interim analysis results until after all monitoring decisions have been made and the database for final analysis has been locked.

The independent DMC will review interim analyses at regularly scheduled intervals, and will make recommendations to the Steering Committee concerning potential modification of the protocol or termination of the trial.

6.1.8 Other Topics

No other topics will be studied.

6.2 Sample Size and Power Considerations

Sample Size for the Entire Trial

Sample size calculations are made for the primary efficacy endpoint, time to first event for the primary composite cardiovascular morbidity and mortality endpoint.

Based on the data reported by major large-scale cardiovascular event trials (e.g. recent ALLHAT¹¹), an annual first-event rate of 3.5% per year for the primary endpoint is assumed for patients in the control group (benazepril+HCTZ). Sample size is calculated with 90% power to detect a treatment difference under the alternative hypothesis of a 15% reduction in risk for the primary endpoint for the Lotrel® treatment group at a two-sided overall significance level of 5%. Considerations for the performance of 4 equally-spaced interim analyses and one final analysis using O'Brien-Fleming group-sequential methods are also made.

To fulfill these assumptions, a total of 1642 patients with a primary endpoint at final analysis are required for both treatment groups combined (except for early completion of the trial for statistically significant interim results). Assuming a recruitment period of 1 to 1.5 years and the corresponding minimum follow-up period of 3.97 years to 3.72 years (total trial duration of approximately 4.97 to 5.22 years), a total of 12,000 randomized and completed patients are required to achieve this number of events. To allow for a rate of slightly less than 5% for patients lost to follow-up, a total of 12,600 randomized patients is planned.

This is an event-driven trial. The trial may be completed early due to statistically significant interim analysis results for the primary endpoint; otherwise, the trial will be completed when a total of 1642 patients with a primary endpoint are obtained.

The actual length of the trial's duration will depend on the observed event rates.

Possible adjustments in the length of the enrollment period and/or the estimated number of randomized patients required to obtain the specified maximum number of patient events may be made during the trial in order to facilitate trial completion within 5 years.

Sample Size for the Ambulatory Blood Pressure Monitoring Subset

The sample size calculation for the ambulatory blood pressure monitoring subset is based on the primary parameters of interest: 24-hour mean ambulatory blood pressures. Assuming a 10% dropout rate, a total sample size of n=280 randomized subjects (i.e. 255 completed subjects) per treatment group is required in order to detect the following differences in 24-hour mean ambulatory systolic and diastolic blood pressures respectively:

Ambulatory Systolic Blood Pressure
a treatment difference (Δ) of 2.5 mmHg with 80% power at two-sided significance level of α=0.05 assuming the standard deviation of ambulatory systolic blood pressure 10 mmHg
a 95% confidence interval of ±1.74 mmHg approximately if there is no treatment difference using a standard deviation of 10 mmHg.

Ambulatory Diastolic Blood Pressure
a treatment difference (Δ) of 2 mmHg with 85% power at two-sided significance level of α=0.05 assuming the standard deviation of ambulatory diastolic blood pressure=7.5 mmHg
a 95% confidence interval of ±1.30 mmHg approximately if there is no treatment difference using a standard deviation of 7.5 mmHg.

The standard deviations are within the range of observed estimates from previous trials.

What is claimed is:

1. A method for reducing cardiovascular morbidity and/or mortality in a mammal with hypertension, comprising co-administering to said mammal:
(a) a compound selected from amlodipine and pharmaceutically acceptable salts thereof; and
(b) an ACE inhibitor selected from benazepril, benazeprilat, and pharmaceutically acceptable salts thereof.

2. The method of claim 1, wherein said mammal is a human.

3. The method of claim 2, further comprising co-administering a diuretic.

4. The method of claim 2, wherein the hypertensive patient is a high-risk hypertensive patient.

5. The method of claim 4, wherein said compound is the besylate salt of amlodipine.

6. The method of claim 4, wherein said diuretic is selected from the group consisting of methyclothiazide, hydrochlorothiazide, torsemide, metolazone, furosemide, chlorthsalldone, N-(5-sulfamoyl-1,3,4-thiadiazol-2-yl)acetamide, triamterene, chlorothiazide, indapamide, bumetanide, amiloride, spironolactone bend roflumothiazide, benzthiazide, cyclothiazide, quinethazone, hydroflumethiazide, polythiazide, trichlormathiazide and ethacrynic acid.

7. The method of claim 2, further comprising co-administering digoxin.

8. The method of claim 2, wherein co-administration is effected for longer than 16 weeks.

9. The method of claim 8, wherein co-administration is effected for longer than six months.
